(19) United States
(12) Patent Application Publication
(10) Pub. No.: US 2002/0068366 A1
(43) Pub. Date: Jun. 6, 2002

(54) Proteomic analysis by parallel mass spectrometry

(76) Inventors: James R. LaDine, Uxbridge, MA (US); Ian Jardine, Los Gatos, CA (US); Mike S. Story, Los Gatos, CA (US)

Correspondence Address: John J. Gagel, Fish & Richardson P.C., 225 Franklin Street, Boston, MA, 02110-2804, US

(21) Appl. No.: 09/835,273

(22) Filed: Apr. 13, 2001

Related U.S. Application Data
(60) Provisional application No. 60/196,889, filed on Apr. 13, 2000.

(51) Int. Cl. G01N 33/543; G06F 19/00; G01N 33/48; G01N 33/50

(52) U.S. Cl. 436/518; 702/19

(57) ABSTRACT

Analysis of, e.g., a proteome, utilizing a parallel array of mass spectrometers.

### CROSS REFERENCE

This application claims priority from provisional application Ser. No. 60/196,889, filed Apr. 13, 2000, the entire contents of which is incorporated herein by reference.

### FIELD

This invention relates to proteomic analysis by parallel mass spectrometry.

### BACKGROUND

Within a typical cell there are several thousand proteins, its “proteome,” which carry out the metabolic work of the cell. These proteins are in constant interplay with one another, and with every other sort of biomolecule found within a cell. The proteins physically interact, or bind, to each other and to common secondary molecules. The result of such interactions is a fine control and balancing of metabolic functions. For example, one protein may increase or decrease the function of another protein by binding to it and altering its structure by the addition or removal of a modifying group such as a phosphate. Another mode of action is for one protein to produce more or less of a secondary substance that interacts allosterically with a second protein (or multiple second proteins) to modulate its function. Analysis of the abundance of proteins can therefore be useful in elucidating the molecular basis of differences brought about by diseases or by therapeutic treatments

A number of techniques have been suggested for analyzing cellular proteins, including, for example, two-dimensional electrophoresis followed by mass spectrometry. In the case of two-dimensional electrophoresis, a protein sample is placed in a gel and subjected to electric fields. The migration of the proteins across and down the gel is dependent in large part on molecular weight and isoelectric point, thus producing a characteristic gel pattern. The gel patterns can be analyzed directly or the protein spots in the gel may be further analyzed by mass spectrometry. Another way of separating proteins prior to mass spectrometry is to apply liquid chromatography of one or more types.

In mass spectrometry (MS), proteins or peptide samples are ionized and the ionized species are subject to electric and/or magnetic fields in a vacuum. From the travel path of the ions, their molecular weights can be deduced. The mass spectrum is a plot of ion abundance as a function of mass-to-charge (m/z) ratio of the ions traveling through the mass analyzer. In one strategy for preparing a sample for analysis, the proteins may be enzymatically cleaved into their constituent peptides prior to MS analysis in order to enhance likelihood that at least some of the protein will be sufficiently ionized so as to be detected. If the sequence of the gene that encodes for a protein is known, a positive identification of a whole protein may be made on the basis of determining the structure of a relatively small piece of the protein using mass spectrometry.

### SUMMARY

An object of this invention is to achieve analysis of a large number of proteins in an accurate, time-effective manner. For example, using liquid chromatography and mass spectrometry in a conventional manner, it may be possible to identify and assign relative abundances to approximately 200 proteins or protein fragments per hour. Those 200 proteins may originate from a single complex sample that is one of several hundred samples queued up for automated analysis. Many cell types have a proteome comprised of approximately 5,000 different proteins, and at present to simplify the analysis, the proteome would typically be fractionated into groups of approximately 200 proteins prior to the liquid chromatography mass spectrometry analysis and identification of the constituent petides arising from those 200 proteins. A proteome of 5,000 proteins could be fractionated into, for example, 36 fractions containing about 140 protein each, or into 25 fractions containing 200 proteins each. At a sample throughput rate of one per hour (a mixture of peptides from 140-200 proteins), the analysis of 36 fractions would take about 36 hours. A single experiment comprised of comparing two cellular states, for example, a drug-exposed state and non-exposed state, over 30 time intervals would generate approximately 2160 protein fraction samples or more. At a rate of peptide analysis, identification, and quantification in the range of about 150 per hour, the comparison would require approximately 90 days to complete. Bearing in mind that there are roughly different 100 tissue types in humans, it would then require about 24 years to characterize the total molecular effect of a drug on all proteins in the various tissues of a human.

Accordingly, in a first aspect, the invention features a method for analysis of proteins in a biological system. The method includes providing a biological system and exposing the system to a stimulus. The biological system is sampled at multiple time intervals after exposing the system to the stimulus. The multiple samples are treated by a separation technique to provide multiple protein samples suitable for analysis by mass spectrometry. The multiple samples are analyzed to determine changes in protein abundance as a function of time after exposing the biological system to the stimulus. The analysis includes providing a parallel array of mass spectrometry systems adapted for protein analysis. Mass spectral data from the mass spectrometry systems in the array is directed to a common computing device. The mass spectral data is indicative of the identity and the abundance of protein in the multiple samples. The mass spectral data is correlated as a function of time.

In another aspect, the invention features a method for analysis of proteins in a biological system including: providing a biological system containing proteins; exposing the biological system to a stimulus; after exposing the biological system to the stimulus, sampling the biological system at multiple time intervals to obtain multiple samples; treating the multiple samples by a separation technique to provide multiple protein samples suitable for analysis by mass spectrometry; providing a parallel array of mass spectrometer systems capable of simultaneous analysis of as many protein samples as there are spectrometer systems in said array; analyzing the multiple protein samples in said parallel array of mass spectrometry systems to generate mass spectral data indicative of the identity and the abundance of proteins in said multiple protein samples; and in a common electronic computing device communicating with each of said mass spectrometry systems, correlating said mass spectral data as a function of time.

In another aspect, the invention features a system for mass spectrometric analysis including a parallel sample separation apparatus adapted to separate multiple samples in parallel for analysis by mass spectrometry and a parallel array of mass spectrometry systems adapted to receive the samples from the separation apparatus. A common computing device communicates with the parallel array of mass spectrometry systems and the parallel separation apparatus. The common computing device to analyzes mass spectral data from the parallel array of mass spectrometry systems as a function of sample identity.

In another aspect, the invention features a parallel array of mass spectrometers and a central computing device. In another aspect, the invention features analyzing multiple samples with a parallel array of mass spectrometers.

Embodiments may include one or more of the following. Correlated data is displayed as a function of protein identity, protein abundance, and time. The correlated data is stored in a searchable database. Proteins are identified based on changes in abundance or a function of time. The array includes 2-5, 4-20, or 15-100 spectrometers. The array may include at least 20 mass spectrometers, e.g., 32 spectrometers. The analysis includes 500 proteins or more, 3000 proteins or more, or 5000 proteins or more. The separation includes a separation apparatus and the common computing device communicates with the separation apparatus. The separation technique includes chromatography, electrophoresis, or magnetic particle separation. The magnetic particle separation apparatus treats multiple samples in parallel. The separation technique is arranged to employ multiple separation schemes on the same sample. The mass spectral data includes peptide fragment mass spectra and an amino acid sequence derived from a data base. The mass spectrometer array includes a liquid chromatograph-tandem mass spectrometer (LC-TMS) mass spectrometer system.

Embodiments may also include one or more of the following. The analysis includes exposing a first component of the biological system to a stimulus and maintaining a second component of the biological system free of the stimulus, sampling and analyzing each of the first component and the second component and comparing the identity and abundance in the first component and the second component. The samples from the first component and second component are analyzed separately. The stimulus is a drug. The time interval is about 5 to 60 seconds. The time interval is about one minute to one hour.

Embodiments may include one or more of the following advantages. Coordinated parallel mass spectrometric analysis of biological samples allows one to analyze samples from a biological source on a time scale that is governed only by the rate of the biological changes one wishes to observe, and not by the rate at which the mass spectrometer performs analyses. A key to identifying proteins of transient activity, but high biological relevance is conducting analyses in relatively short time intervals. Studies of massive numbers of proteins in short time intervals can be achieved accurately and in a time effective manner by employing a coordinated array of mass spectrometry systems.

By identifying the time-order of protein-related cellular changes, one may infer the order of interactions between and among proteins. The approach requires no advanced knowledge of pairs of interacting proteins, such as would be gained by protein interaction experiments. Further, all protein interactions occur in vivo, in their proper subcellular compartments, in the presence of proper concentrations of cofactors, substrates, and metabolic fuel. Thus the potential for artifactual and false observation of protein interactions that occur in vitro is necessarily reduced. The approach may also provide for simultaneous recognition of multiple protein interaction pathways and their points of intersection. That is, a protein whose function sits at a branch point in a plurality of metabolic pathways relevant to a disease-state may be recognized as such without any foreknowledge of the proteins or pathways likely to be involved. The involvement of a protein in multiple metabolic pathways has significant implications on its desirability as a target for drug intervention, or as a diagnostic target. One would, a priori, desire a protein target of drug action to have minimal co-involvement in nondiseased state metabolism.

Another benefit of time-resolved analysis of total cellular protein is that the time dependent appearance and disappearance of protein in normal cells compared to a cell that is treated with drug or perturbed by a disease or other factor can be determined. In this case, the proteins involved in that perturbation would be revealed. The ability to see a large number, even all, or essentially all, proteins involved in the drug action pathway, that are the target of drug action, and the ability to determine involvement of any protein in that pathway and other unforeseen pathways would be highly desirable in selecting alternative points of drug action in cases where drugs have undesired reactions.

A time-dependent, time-resolved study of proteomes may reveal not only increases and decreases in the abundance of particular proteins over time, but will also reveal shifts in structural state of those proteins with the total abundance. For example, the total concentration of an enzyme might not change in response to a stimulus, but it may become modified chemically to a greater or lesser degree during that response. A shift in the balance of structural states may occur with or without a concomitant change in a particular protein's total abundance. The system and method may identify points at which protein modifications have occurred, and reporting the degree of modification of any protein. The system can be adapted for analysis without admixing perturbed and unperturbed cell fractions or samples.

All publication and patent documents referenced herein are incorporated by reference in their entirety. Some references are referred to by author and year. These references are identified in the appendix at page 29.

Still further aspects, features, and advantages follow.

### DESCRIPTION OF PREFERRED EMBODIMENT(s)

We first briefly describe the drawings.

### DRAWINGS

FIG. 1 is a schematic of an analysis of a biological system;

FIG. 2 is a schematic of a parallel mass spectrometry system;

FIG. 3 is a more detailed schematic of the data and control connectivity of a system utilizing multiple magnetic particle separation systems for sample processing and multiple LC-MS systems for sample analysis;

FIG. 4 is a schematic of the system in FIG. 3 illustrating physical arrangement of a system for sample transfer;

FIG. 5 is a more detailed illustration of mass spectrometric analysis utilizing LC-TMS;

FIG. 6 is a schematic of a central computing device; and

FIG. 7 is a flow diagram of the central computing device operation.

### DESCRIPTION

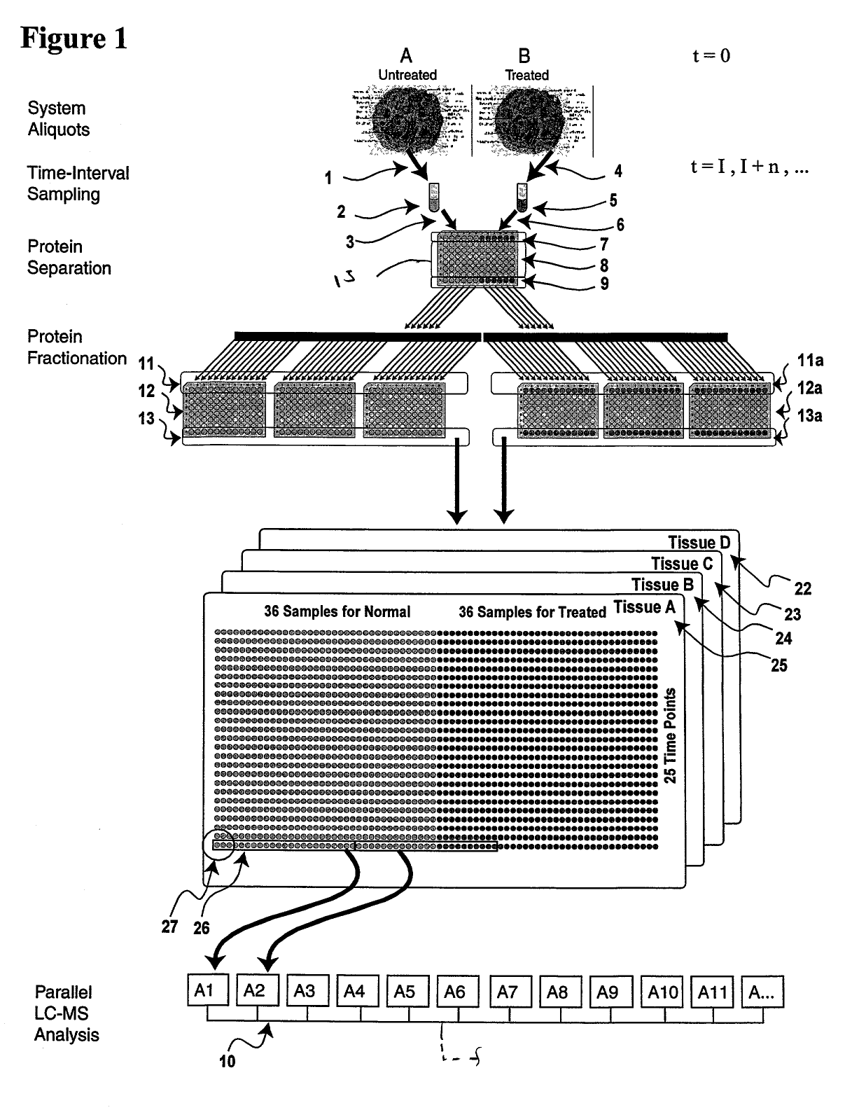

Referring to FIG. 1, an analysis of a biological system may include providing two aliquots of the system, aliquot A and aliquot B. The biological system may be, for example, a type of cell, for example, representing a tissue type. The samples may be stored in a medium in which the cells remain viable and metabolically active.

At a time t=0, the cells in aliquot B are perturbed, for example, by exposure to a test influence such as application of a drug candidate to the cell culture. At time intervals t=I, I+N, . . . a sample of cells 2 is removed 1 from aliquot A and a sample of cells 5 is removed 4 from aliquot B and treated to produce raw lysate samples. This process is repeated for the desired number of time intervals. The lysate samples may be placed 3, 6 in sample holders, e.g., by an automated, computer controllable device such as robotic pipette. In the embodiment illustrated, the sample holders are the wells of a microplate 15 that is used in a parallel magnetic particle separation apparatus, which will be described in detail below. Briefly, the wells are held in a well tray or microplate 15. Each raw lysate sample from aliquot A and aliquot B is divided into six portions placed into six wells in the first row 7 of the microplate 15. The samples are treated by magnetic particle separation to separate and wash proteins using the wells in rows 8, 9. For example, the separation may be according to subcellular location or gross physicochemical characteristic. In this illustration, samples at each time interval are provided in six wells so that up to six different separation schemes may be coordinated in parallel.

Next, the separated protein samples are replicated into multiple new plates, and the proteins are re-fractionated, e.g., by selection using multiple second dimensions of interaction with moieties on the surface of a solid support. In the embodiment illustrated, the fractionation also is also carried out using magnetic particle processing. The separated samples are divided in the wells of the first rows 11, 11a of multiple trays. The wells in rows 12, 13, 12a, 13a are used for fractionation. In this illustration, each of the six separated subsamples from the protein separation stage is divided into six wells for further fractionation using duplicative or alternate strategies. The subsamples may be divided and transferred using a computer controlled device such as a robotic pipetting station.

The number of subsamples that may be produced by this process from 25 time intervals for each of four tissue types 22-25 is illustrated. The number of these subfraction samples that a single mass spectrometry system may typically analyze in one day, assuming a level of complexity of about 200 proteins per subfraction sample, is indicated by box 26.

The peptide mixture subsamples are subject to mass spectrometric analysis using mass spectrometer system that is in a coordinated array 10 of multiple mass spectrometry systems which analyzes samples in parallel. Using the desired number of time intervals, the identity and relative abundance of each protein, as determined by mass spectrometric analysis, is collated as a function of time. As a result, the abundance profile for a large number of proteins as a function of time after perturbation can be determined. The subsamples may be analyzed as soon as they are separated and fractionated. Alternatively, analysis may be conducted after all of the samples from all time intervals have been separated and fractionated. The samples may be transferred to the spectrometer systems using a computer controlled robotic sample handler.

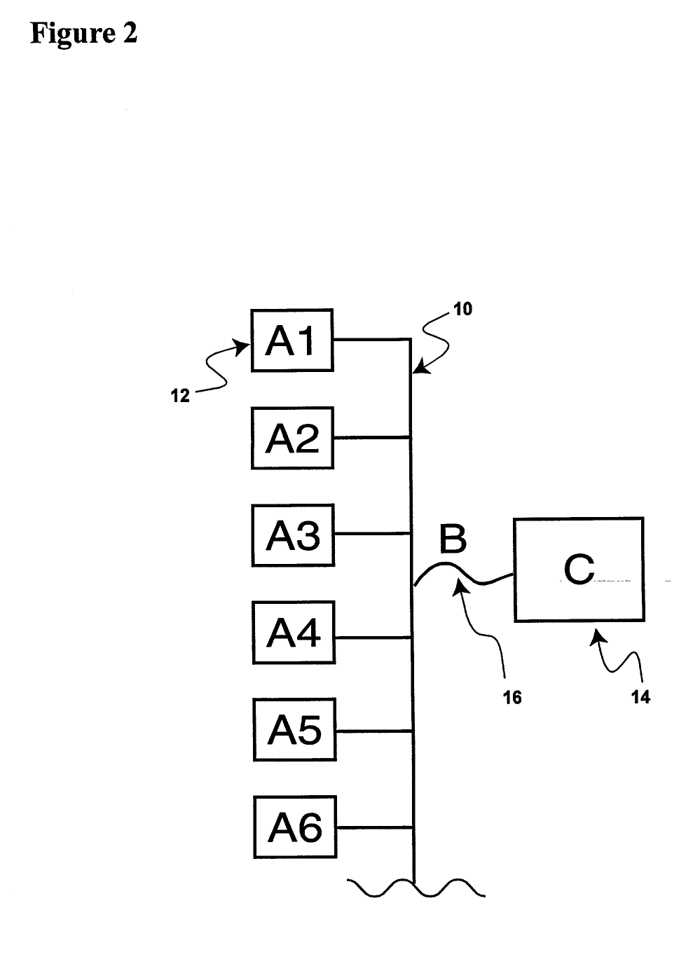

Referring as well to FIG. 2, the system 10 for conducting the mass spectrometric analysis includes an array of mass spectrometry systems 12, in this example six spectrometers are shown (A1-A6), and a central computing device 14. The central computing device is connected to the spectrometers by a data link 16. Each spectrometer in the array 12 of mass spectrometry systems may conduct analyses simultaneously. As a result, as many samples as there are spectrometers may be simultaneously analyzed. These spectrometers may be controlled by the central computing device 14 via the data link 16. In addition, the mass spectral data, representing the abundance and identity of proteins in the various samples is transmitted to the central computing device 14 via the data link 16. The central computing device then automatically collates the data from the spectrometer array as a function of time so that protein abundance as a function of time may be determined and displayed on the display device.

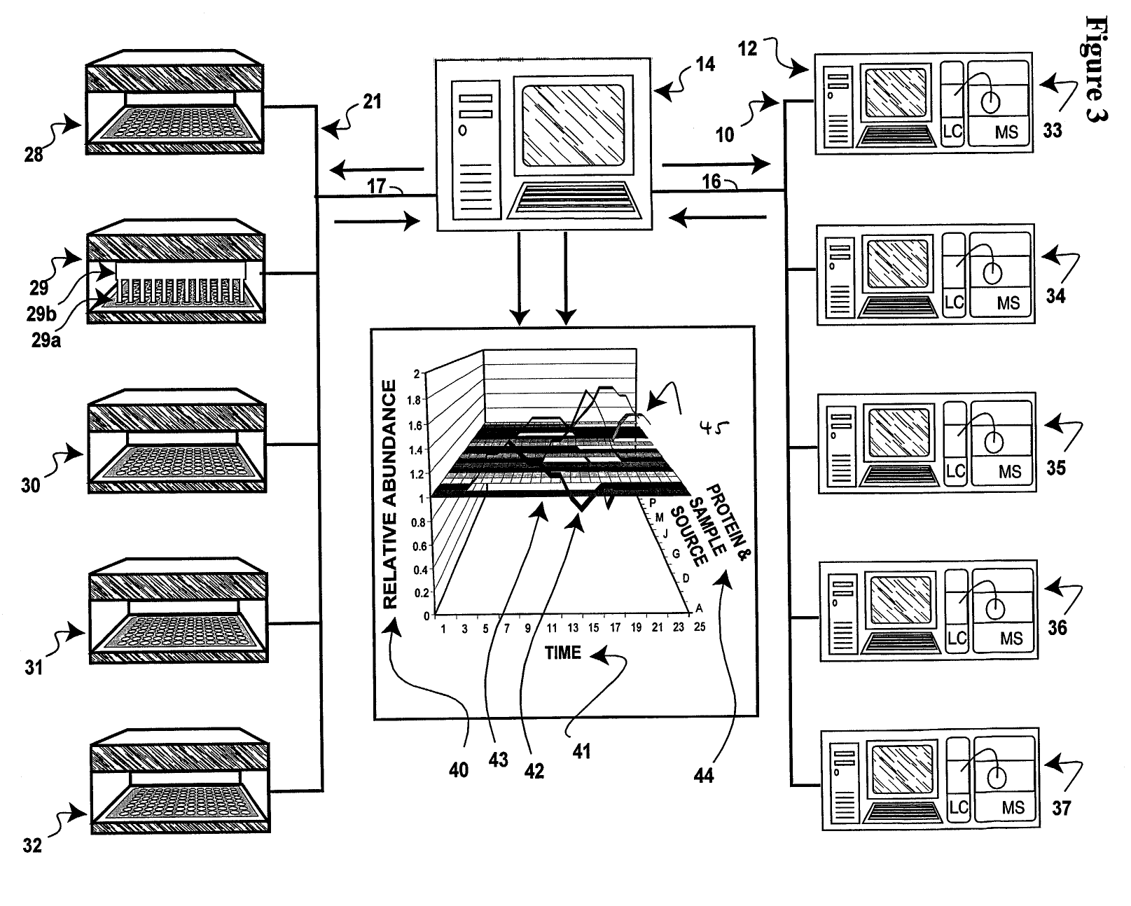

Referring as well to FIG. 3, the system 10 is illustrated in more detail to include an array 12 of mass spectrometry systems 33, 34, 35, 36, 37 and, in this embodiment, an array 21 of sample preparation devices, i.e. magnetic particle separation devices 28, 29, 30, 31, 32. The arrays communicate through data links 16, 17, with the central processor 14, which sends control information to direct the function of any sample separation/fractionation device or mass spectrometry system and receives back sample identity and sample analysis results for collation. The separation devices and mass spectrometry systems in the array may be of several types but are preferably chosen in coordination such that sample treatment and mass spectrometry analysis can be carried out in a parallel manner. As a result, the separation device or devices preferably provide for multiple different selective separations in parallel. The preferred separation device is a parallel magnetic particle type separation device that treats multiple samples in parallel, as discussed in more detail below. The mass spectrometer type is preferably a tandem mass spectrometer coupled to a liquid chromatograph (LC-TMS).

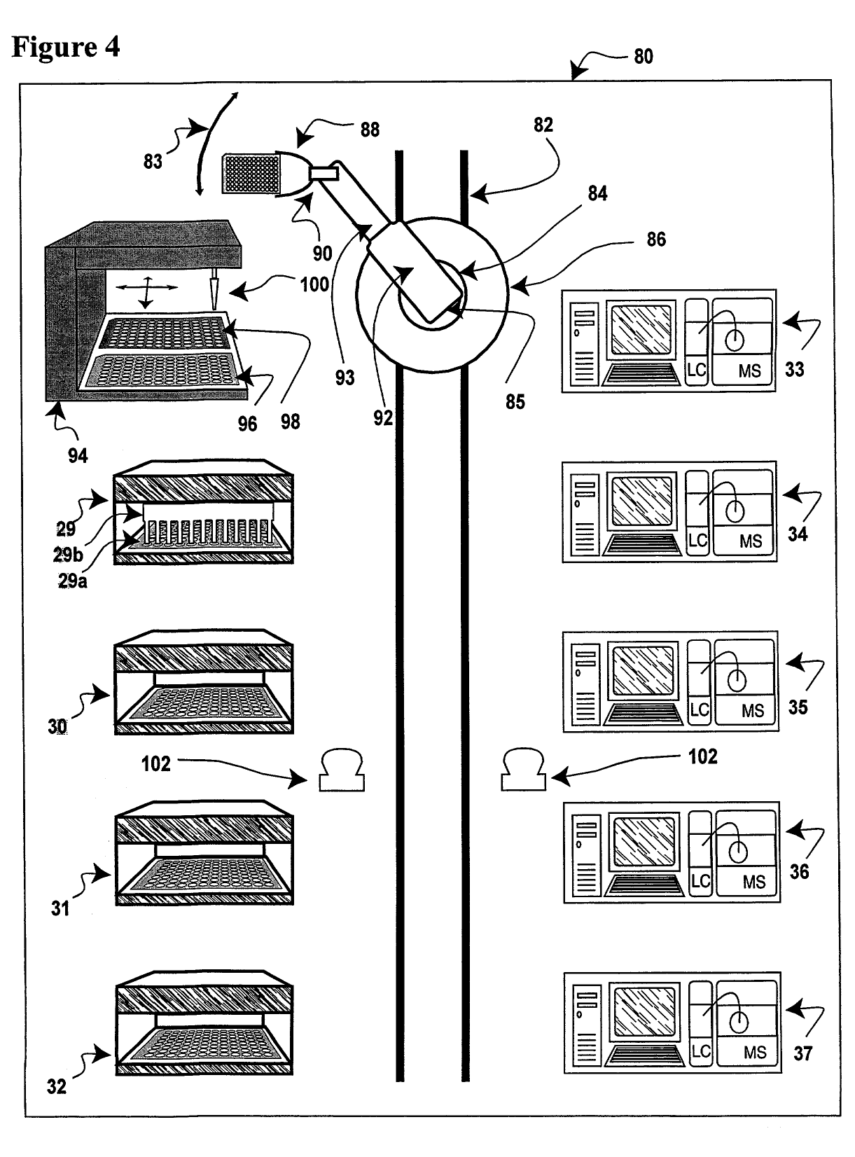

Referring to FIG. 4, the movement of samples between elements in the system 10 is illustrated. The system is mounted on a bench 80 upon which separation apparatus 30, 31, 32, the parallel array of spectrometer systems 33, 34, 35, 36, 37, and an automated liquid dispensing device 94 are arranged around a rotating robotic arm 92. The robotic arm may move along a rail 82 that is also mounted on the bench 80. The arm includes a grasper 88 which can grasp trays of sample wells. The grasper pivots (arrow 83) around a wrist joint 90. The arm 92 may extend to various distances and positions in any direction along the rail mount by the action of knee joints 85, 93 and swivel joint 84. The motions of the robot arm are controlled by the central computer 14 (FIGS. 2, 3, 6).

The dispensing device 94 includes at least one dispenser 100 and is configured to, for example, dispense reagents and lysates from the wells of a first reagent tray 98 into the wells of a second tray 96. The arm then grasps the tray 96 from the dispenser 94 and positions it at a magnetic particle separator (e.g. 29), where the sample is treated as discussed above. After separation or fractionation treatment, the robotic arm 92 then positions the tray at an LC-MS system in the array. Alternatively, the tray may be returned to the liquid sampling device to transfer sample to another tray which is then moved to a mass spectrometry system. The LC-MS system includes an autosampler. The motion and identity of moving trays is tracked by one or more devices such as bar code scanners 102. In other embodiments, sample transfers may be done manually.

The following discussions further describe certain embodiments.

Separation and Fractionation

Referring to FIG. 3, the separation system is preferably a magnetic particle separation system. A magnetic particle system 29 includes an array of sample wells 29a in which multiple samples may be placed. An array of magnetic probes 29b extending across the rows of wells and positioned above the wells selectively moves the particles into adjacent rows of wells for processing samples. In magnetic particle separation system, magnetic particles coated with a specie capable of binding with a desired molecule type are deposited into wells containing biological sample. The particles which gather the desired molecules by binding are then removed by introducing the magnetic probe into the wells. The particles can then be deposited in subsequent wells where additional processing steps, such as digestion, washing, etc. can be carried out, thus substantially simplifying the samples by removal of the desired material, in this case protein or certain proteins, from raw biological sample. Magnetic particle based sample fractionation can be performed, for example, on whole Eukaryotic cells (Kvalheim, Fodstad et al. 1987; Jackson, Garbett et al. 1990), prokaryotic cells (Islam and Lindberg 1992), viruses (Ushijima, Honma et al. 1990) membrane fragments (Bennick and Brosstad 1993), liposomes (Scheffold, Miltenyi et al. 1995) cell organelles (Owen and Lindsay 1983), phage particles (Gebhardt, Lauvrak et al. 1996) soluble intercellular proteins (Kandzia, Scholz et al. 1984) and nucleic acids (Ozyhar, Gries et al. 1992), and cellular metabolites (Dieden, Verbeeck et al. 1999). This variety of samples spans broad levels of sample complexity and molecular size. Magnetic particle separation is preferred because multiple samples can be processed and prepared for analysis quickly and in parallel, and may be applied to the fractionation of soluble and insoluble biochemical components, thus enabling multiple dimensions of parallel fractionation upstream of analysis by a parallel array of mass spectrometers. A more detailed discussion of magnetic particle separation is provided in Tuunanen U.S. Pat. Nos. 6,040,192, 5,942,124, 6,020,211, 5,647,994, and U.S. Pat. Ser. No. 09/646,204, filed Sep. 14, 2000, the entire contents of all of which is incorporated herein by reference. A suitable system is the Kingfisher, available from Thermo Labsystems, Helsinki, Finland, which includes up to twelve magnetic probes operating in parallel. A suitable liquid dispensing apparatus is the Well Pro, also available from Thermo Labsystems. A suitable robotic handler is the CRS Handler (Ontario, Canada).

Referring particularly to FIG. 1, lysate samples from aliquot A and B can be placed in the wells of upper row 7, which include magnetic particles derivatized with the desired moieties (e.g. antibodies, reactive groups, streptavidin). In this illustration, six wells are provided with sample taken at each time interval so that separation strategies can be replicated or multiple different separation strategies can be conducted in parallel. Middle rows 8 are filled with beads, washing buffers, and bead re-collection buffers. The wells of final row 9 are the destinations for the separated (simplified) samples. Collected material from the wells of row 9 can then be distributed to the starting position of new microplates 11, 11a for multiple second dimensions of fractionation. Beads, buffers, and other reagents for this fractionation may be contained in the central rows of these plates 12, and the final row 13, 13a is once again used as the row where subfractionated proteins are deposited for further processing and analysis. Further processing may include a number of methods which can be used to prepare a mixture of proteins of high complexity for mass spectrometry. (Yates, McCormack et al. 1997; Link, Eng et al. 1999; Yates, Carmack et al. 1999; Gatlin, Eng et al. 2000) In a preferred method, the mixture of proteins is treated with a protease (trypsin) to cut each protein into a large number of peptides. These peptides are separated by two-dimensional liquid chromatography prior to electrospray ionization and tandem mass spectrometry analysis of mass and relative abundance, as discussed below.

Additional methods to fractionate a proteome in this manner prior to analysis are also possible. For example, if a particular cell type contained 5000 proteins (the proteome), then the separation may allow that proteome to be made into 25 groups of 200 proteins each. Amenable separation technologies to do this include those based on sorting proteins according to:

1. Their molecular size and molecular isoelectric point (charge properties). This technology is embodied in a 2-D gel method. Examples are in (Cordwell, Nouwens et al. 2000; Corthals, Wasinger et al. 2000)

2. Their amino acids content and reactivity characteristics of those amino acids. (Aebersold, Rist et al. 2000; Spahr, Susin et al. 2000)

3. Their degree of modification with chemical moieties such as phosphorylation, glycosylation, sulfation and the degree to which those proteins bearing those groups may be sequestered on the basis of chemical reaction or affinity capture. (te Heesen, Rauhut et al. 1991; Zhang, Czernik et al. 1994)

4. Their adherence to, or incorporation into, membranes. (Santoni, Doumas et al. 1999; Santoni, Rabilloud et al. 1999; Morel, Poschet et al. 2000; Simpson, Connolly et al. 2000)

5. Their solubility. (Taylor, Wu et al. 2000)

6. Their degree of incorporation into macromolecular complexes that may be sequestered by affinity capture methods or by centrifugation. (Hayden, McCormack et al. 1996; Saleh, Schieltz et al. 1998; Link, Eng et al. 1999; Panigrahi, Gygi et al. 2001)

7. Their degree of incorporation into subcellular structures (organelles) that may be isolated using affinity capture methods or centrifugation. (Meeusen, Tieu et al. 1999; Cordwell, Nouwens et al. 2000; Morel, Poschet et al. 2000; Taylor, Wu et al. 2000)

8. Separation by liquid chromatography based on differentiated affinity with a selective solid support.

While the preferred separation apparatus is a magnetic particle separation apparatus, other techniques such as a liquid chromatography may also be used. There is a trade-off between the number of fractions created and the degree of complexity in their protein content. Whatever method is used to make the subfractions, for whole proteome analysis, the number of fractions multiplied by the number of proteins in each fraction should typically contain approximately at least the total number of proteins itself (5,000 in this example).

In addition, the subfractionation of cellular contents by a single technique prior to proteomic analysis can lead to “blind-spots,” i.e., parts of the proteome are not easily captured in a category and lost to analysis associated with each approach. It is desirable, therefore, that the fractionation regime incorporate at least two different and complementary strategies for fractionation. One such dual fractionation strategy might be to fractionate according to one physicochemical characteristic (i.e. molecular size) and one biological characteristic (i.e. organellar association). As a practical matter this doubles the number of samples required for exhaustive analysis of an entire set of cells. Data regarding the separation technique is tracked by the central computing device.

In addition, the fractionation stage may include methodologies to compensate for peptides from a given protein that may not be suitably ionized for mass spectrometry analysis. For example, typically 20 percent of the linear amino sequence of a protein is ionized and analyzed by tandem mass spectrometry. The other peptides are simply silent in this analysis. Because of this, interesting peptides bearing sites of modification may be missed in the overall analysis. To remedy this potential problem, multiple dimensions of cell sample fractionation may be employed, wherein one of the fractionation methods selectively pulls out, or enriches, proteins or peptides bearing modifications so as to increase the likelihood that they will appear in the final analytical result. Several kinds of such enrichment are discussed in (Soskic, Gorlach et al. 1999; Charlwood, Skehel et al. 2000; Yanagida, Miura et al. 2000).

Mass Spectrometry

Referring to FIG. 3, a preferred type of mass spectrometry system for use in the array 12 is an LC-TMS system, which includes a liquid chromatograph that provides an additional stage of sample separation, which is followed by analysis by tandem mass spectrometry. The system may include its own computing device to operate the function of the mass spectrometer and chromatograph and analyze mass spectral data, which is communicated to the central processor 14.

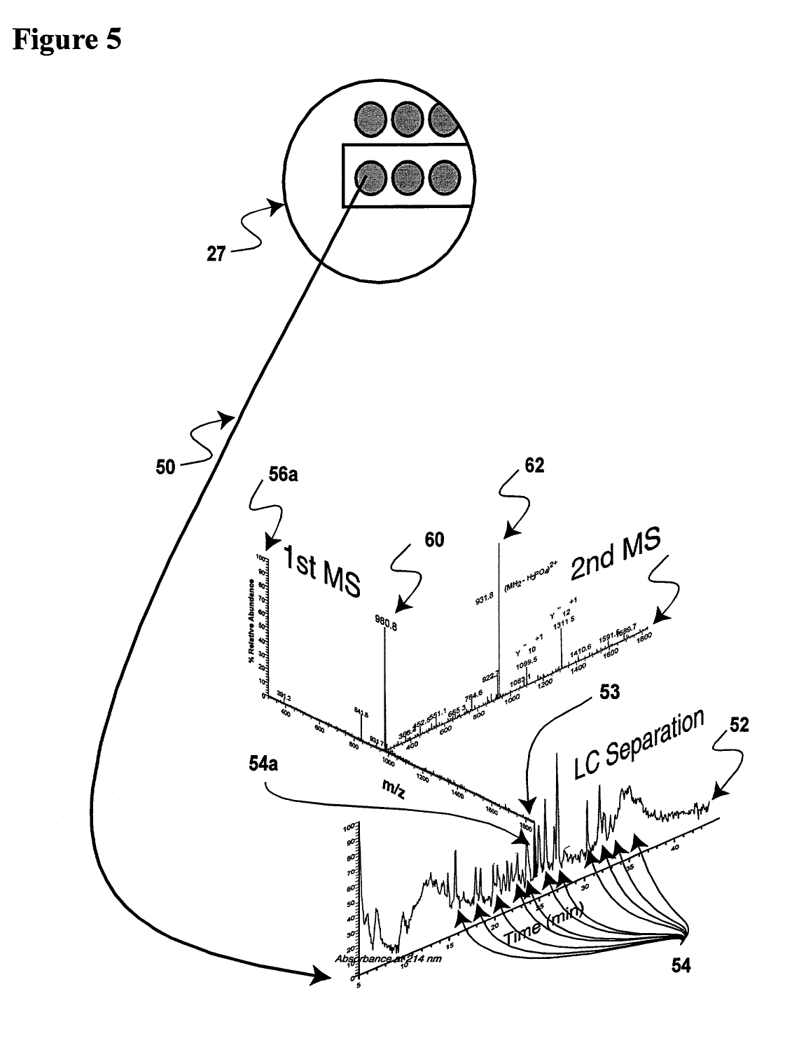

Referring to FIG. 5, the analysis of a single subfraction sample is illustrated. As discussed above, as a step in the fractionation, the protein content of a sample can be reduced and alkylated, then enzymatically cleaved into its constituent peptides in successive steps in a magnetic particle device. This peptide mixture is then be injected into an LC-TMS system whereupon the peptides are separated chromatographically 52 to provide a chromatogram 52 whose peaks 54 indicate eluted peptides 54. For a given peptide such as peptide 54a, a first stage of MS will generate a mass spectral measurement of the abundance 56a of the ion of that peptide and the m/z 53 of that peptide. A second stage of MS may be used to generate multiple subfragments of that peptide ion 60 so as to produce certain of its characteristic subfragments, e.g., 62, which increases the certainty of peptide characterization.

The process of performing tandem MS characterization of peptide identities is described by Yates et al., U.S. Pat. No. 6,017,693, the entire content of which is incorporated herein by reference. This method typically utilizes the known genome sequence for the organism being characterized so as to make the automated comparison between fragmentation patterns that are observed with those that may be predicted on the basis of the gene sequences. A preferred mass spectrometry LC-TMS system is available as the LCQ Deca XP from Thermo-Finnigan Corporation LLC, San Jose, Calif. Interpretive software is provided for use with the mass spectrometry systems(e.g. Sequest available form Thermo-Finnigan Corporation LLC, San Jose, Calif.) to map each observed peptide back to an overall protein sequence from which it came. By summing the relative abundances of the component peptide masses, one may arrive at a number that may be used to describe the abundance of that particular protein relative to the abundance of other proteins in that cellular sample taken at that particular time and circumstance.

In other embodiments, other types of mass spectrometry systems can be used including systems that do not include a separation device such as a liquid chromatograph as in the LC-MS system described above. Higher orders of MS analysis may also be used, for example MSⁿ to provide for de novo sequencing of peptides whose sequences do not reside in a genomic database. By further sample simplification the number of proteins or peptides in a sample may be reduced to a number that may be analyzed by a single stage of MS analysis such as MALDI-TOF. The array may also include different types of spectrometers which are used selectively based on sample capability.

The number of spectrometer systems in the parallel array is selected to effectively analyze the number of samples produced by the separation system. Preferably, the number of spectrometers is the same as the number of separation apparatus, but this is not necessary. In the case of a parallel separation apparatus, there may be more spectrometers than separation apparatus. For analysis of 3-5000 proteins, the number of spectrometers is preferably 6 or 10 or more, more preferably, 20 to 25 or more. The separation apparatus may be a liquid chromatograph coupled to the mass spectrometers (LC-MS), without substantial upstream processing, in which case the number of separation apparatus could be the same as the number of spectrometers.

Biological System Pertubations

As discussed above, the protein abundance may be utilized to study pertubations on a cellular system. One example of a perturbation is exposure of a cellular system to a drug candidate. The drug candidate may be a small molecule, a hormone, a peptide, a protein, a nucleic acid or a plurality of such molecules. Other pertubations include exposure to heat, light, cold, motion, agitation, exposure to cellular material from other tissues, organisms, or microorganisms or cellular systems that have a disease.

The duration of time intervals at which the biological system is sampled may vary and can depend on the time scale of the gross physiological change that occurs in response to the stimulus. For example, one may wish to observe the effect of a drug like aspirin over its minute-scale course of action. Alternatively, a longer acting stimuli, such as exposure to a steroid hormone that requires weeks to bring about its effect, may be studied over a commensurately longer time course. However, even for long acting stimulus, short term proteomic changes may be studied. Typically, the time interval is short compared to the time needed to separate, fractionate, and/or analyze the samples by mass spectrometry. Typical time intervals are about 5 to 10 seconds or about 30 to 60 seconds or about one to about ten minutes. Other intervals include on the order of hours or days. As discussed above, in preferred embodiments, the entire proteome of a cell type is analyzed; however, the system and techniques described herein can be used for analysis of less than the entire proteome. Preferably, the system is arranged to analyze about 500 or more, more preferably about 3000 or 5000 or more, proteins. In addition, the proteins may be derived from disparate sources, such as different cell types, rather than the same cell type. Further, species other than proteins, e.g., nucleotides or other biological molecules, can be analyzed. The parallel mass spectrometry array can be used to analyze any large collection of samples whether of biological origin or some other origin, e.g., environmental samples. While it is preferred that perturbed and unperturbed samples remain physically separate through the separation, fractionation and analysis, the samples may be isotopically labelled and combined prior to any of these stages, e.g., as discussed in WO 00/67017.

Hardware and Software

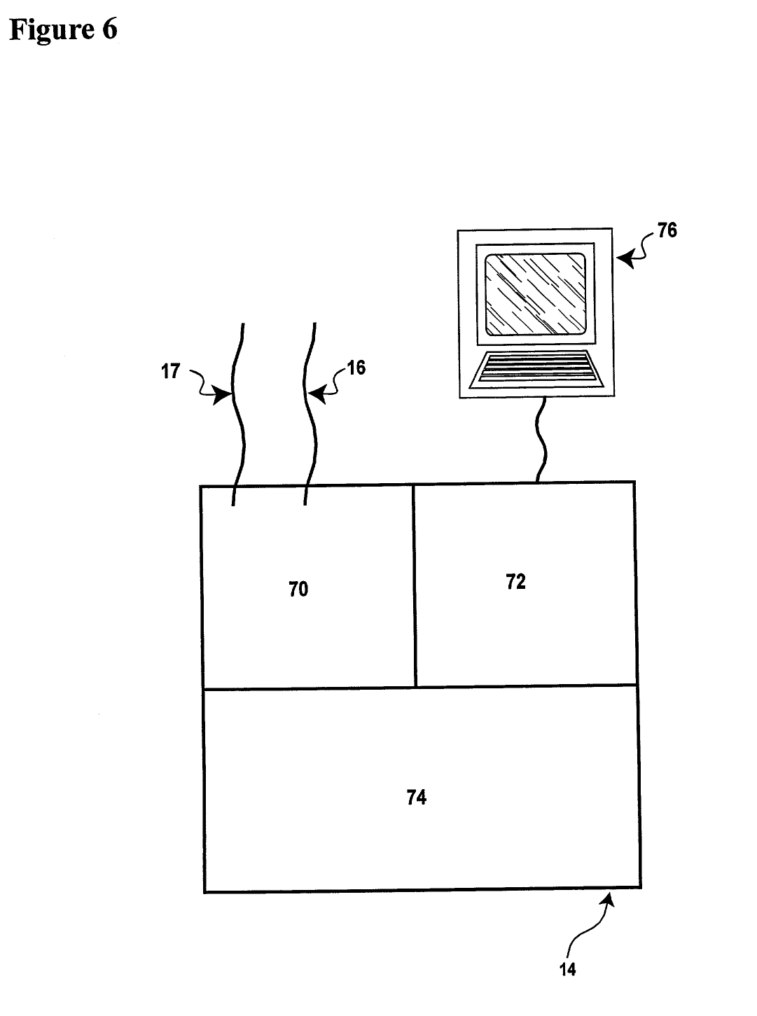

Referring to FIGS. 3 and 6, the central computing device 14 includes a communication module 70, a storage module 72, and an analysis module 74 and a display 76. The communication module 70 is adapted to send and receive data and instructions regarding the parallel array of mass spectrometry systems and the separation devices. The storage module 72 provides for data storage, including storage of mass spectra and time interval information corresponding to the mass spectral data. The analysis module 74 is adapted to analyze the data generated by the experiment. For example, the module 74 collates the mass spectral data and/or protein identities and abundance values as a function of time. The analysis module 74 may also be adapted to analyze mass spectral data to determine the identity of proteins based on the mass spectral data. For example, the analysis module may utilize the technique described in Yates et al. U.S. Pat. No. 6,017,693. The data can be displayed and manipulated on a display device which may include a keyboard for user communication with the central computing device.

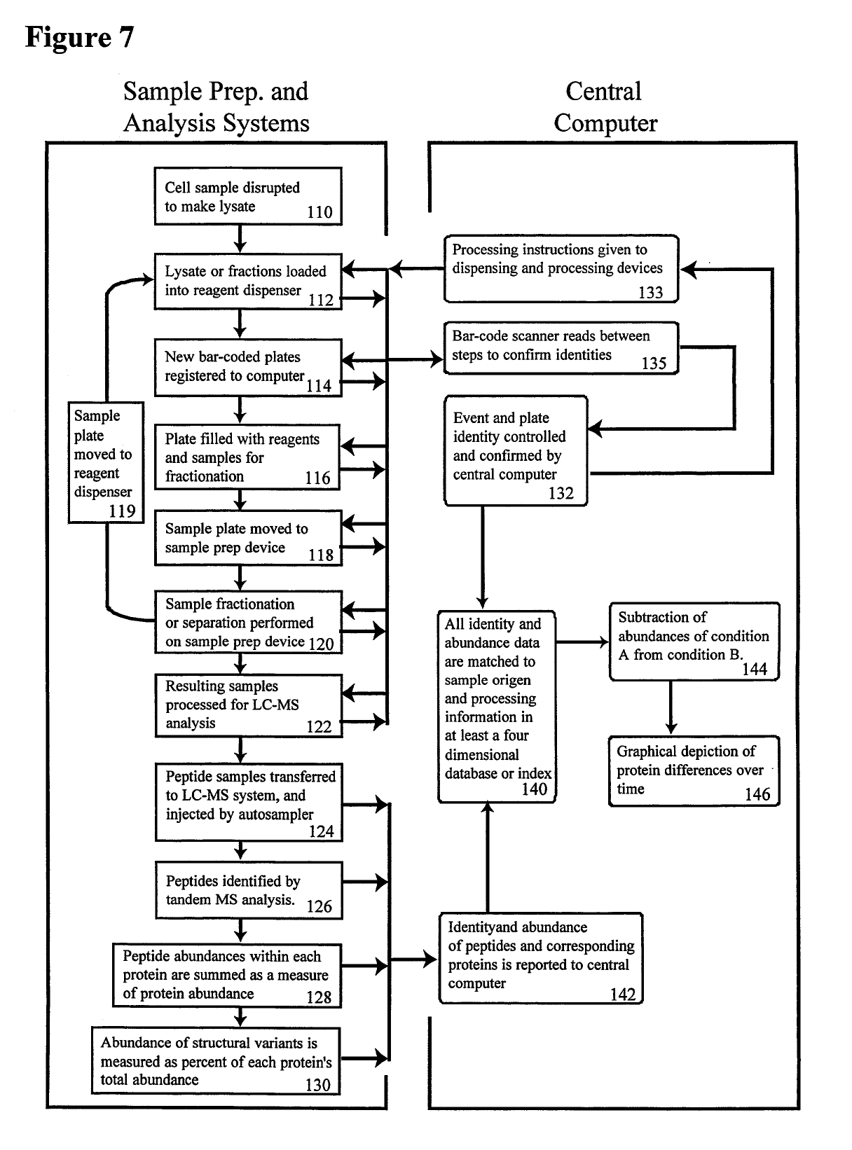

Referring to FIG. 7, a flow diagram illustrate the function of the computing device during an analysis. As discussed above, the cell sample is disrupted to make lysate 110. The lysate is manually loaded into a reagent dispenser 112. The computer then instructs 133 the robot to locate a bar coded plate at the dispenser and registers the plate to the computer 114. A user may enter information into the central computer regarding the separation or fractionation strategy of the plate and the time interval of samples in the plate.

The central computer then instructs 133 the reagent dispenser to fill the plate wells with reagents and samples needed to carry out the strategy 116. The filling of the plate is reported to the computer 132. The computer then instructs 133 the plate be moved to an available separation device 118 and the movement of the plate is reported to the computer 135, 132. The computer instructs 133 the separation device to conduct the separation and the separation is reported 132. For additional processing and fractionation, the computer instructs 133 that the plate be moved back to the reagent dispenser 119.

If the fractionation is complete, the samples are prepared for MS analysis, e.g., by typsinization, which may be conducted in the fractionation well tray or done in a separate well tray 122. The computer then instructs 133 that the tray be transferred to an available mass spectrometer system in the array 124. The computer instructs 133 that the mass spectrometer system begin analysis of the samples, including separating the samples by liquid chromatography 124, identifying peptides by mass spectrometers 126, determining peptide abundances 128 and abundances of structural variants 130. This data is reported to the central computer 142.

The central computer then matches the abundance and identification data with sample origin and processing information 140. The central processor subtracts abundances of proteins in the unpeturbed and perturbed sets and stores the data 144. After multiple samples sets have been analyzed from different time intervals, a graphical depiction of protein abundance differences is produced 146.

Referring particularly to FIG. 3, through data interconnections, a database of at least four parameters is automatically created, those parameters are: time after stimulation of the cell samples 41; the relative abundance of protein observed as, for example, the sum of constituent peptide abundances 40; protein identity for which peptide constituents are summed 44; and whether or not the cell sample withdrawn at the beginning was from condition A or condition B (i.e. perturbed or not perturbed). The data analysis module of the central computing device may perform subtraction of any or all data observed for condition A from all data observed for condition B, or vice versa. The result of this subtraction is a three-parameter representation of only the points of difference between condition A and condition B may be produced. In the preferred embodiment, protein identities showing no change over time 43 between the two conditions may be eliminated from view, and protein identities may be obtained by selecting visually obvious points of increase 43 or decrease 42 in differential abundance.

The computerized instructions and hardware for a multipart experiment distributed among a plurality of liquid chromatography—mass spectrometry instruments tracks experimental samples and subsamples according to an overall index (map) of sample sources, sample identities, sample locations, instrument identities, protein identities, protein abundances, and protein sub-structure abundances. The data resulting from a given instrument on a given sample will be automatically submitted via a hardware connection to the central computing device containing the index, and specifically to its proper data-cell within that index.

In one embodiment, the multi-instrument coordination of sample processing for the aforementioned software and hardware does the following.

1. A model of the overall experiment is constructed. The model encompasses the various kinds of biochemical samples consumed and generated at each preparative and analytical step of the overall experiment.

2. Automatically readable identities are assigned to the sample containers, i.e. the various microplates and tube racks that are generated. At each instrument, the identity of any sample is automatically read and affixed electronically to the results that are generated from that sample on that instrument system.

3. An efficient is established for coordinating the application of each of those racks and plates to the various sample processing and sample analysis instruments based on e.g., the availability of mass spectrometry systems in the array.

4. The identity data output from each individual LC-TMS run is gathered and an index of protein identities, and protein structural states, that are tracked over the various times and conditions of the experiment is created.

5. It assigns to each indexed protein the abundance of that protein observed under each condition and time. For example, the abundance value may be acquired as the sum of the integrated LC-MS areas of all of the daughter peptide ions produced from each protein.

6. It assigns to each indexed protein, the proportion of it existing in a modified state. For example, this value may be acquired by tracking the relative integrated LC-MS areas of only the daughter ions representing the peptides spanning sites of detected modification.

7. It monitors the abundance of internal standards that may be applied across all samples and normalizes the whole of the protein abundance data on the basis of the abundance of the standards. Metabolic proteins of little significance to metabolic regulation (“housekeeping proteins”) such as Hexokinase, 3-Phosphoglycerate Kinase and Glyceraldehyde Phosphate Dehydrogenase have recently been described as useful proteins to track the reproducibility of processing from sample to sample in a multi-part proteomic analysis (Thompson, P. Oral presentation at 2000 CPSA meeting, Princeton, N.J.). These proteins are essential to basic cellular respiration and tend to be expressed and represented in the proteome in a stable fashion on a perviable cell basis

8. It subtracts all of the identity and abundance results generated over time for one cell type or condition from all of the analogous identity and abundance results generated over time for another cell type or condition, and construct a graphical depiction of this multifold difference.

### EXAMPLE

The following is an example of how a time-resolved proteomic analysis is achieved. Each methodological step will be described (1), followed by a more detailed description of each step (2) including discussion of the magnitudes of samples, coordination, and data management. This example discloses:

1. A process wherein:

a. An experiment involving identification and quantitation of all proteins contained within two kinds (or more) of cells is designed. This experiment is designed to make the measurement of all proteins from the cells at a number of different times, and in a number of different cell types. Such an experiment requires many hundreds of sample processing vessels, sample processing robots, and several analytical systems capable of LC-MS. The identities of all vessels, devices, and instruments are known to a central computer so that data from any vessel will be properly recorded

b. Cell samples are withdrawn from cell culture or living tissue at predetermined intervals.

c. Those cells are disrupted so as to burst the outer cell membrane and spill the liquid and nonliquid components of the cells into a common mixture called a lysate.

d. Cell lysates may each contain approximately 5,000 different proteins. The lysates are fractionated into, for example, five fractions, each of which would contain approximately 1,000 different proteins. This fractionation may be done by using magnetic particle separation or other means for separating proteins on the basis of their charge, solubility, hydrophobicity, and association with macromolecular structures that may be obtained by other means. This fractionation may be repeated to generate greater numbers of subfraction, each containing a commensurately lower number of total proteins on average.

e. The protein subfractions are then treated with reagents such as reducing agents, alkylating agents, and other chemicals that react specifically with various amino acids on the proteins.

f. The mixtures of proteins of each subfraction are then digested into several constituent peptides using trypsin or some other protease.

g. The samples containing mixtures of peptides are then separated by one or two dimensions of liquid chromatography and then mass analyzed by single or tandem mass spectrometry.

h. The peptide masses and fragment masses are then compared to databases of predicted peptide masses and fragment masses to determine the most likely sequence of each peptide. From this sequence, the identity of the protein of origin may be established.

i. The identity of each peptide identified in this manner, from each of the hundreds or thousands of pre-analytical samples generated in this experiment, is relayed to the central computing device that is configured to match the peptide identity and quantity information that it receives with the information that it receives about sample identities and physical locations during the execution of the sample preparation steps of the experiment. That is, the peptide data from a sample is always linked and matched to the identity and history of the automatically-generated sample vessel from which it came.

j. The abundance of each peptide discovered in the first cell type (e.g., healthy, or condition A) is then compared to the abundance of each peptide discovered in the second cell type (e.g., sick, or condition B). This is done by alignment of identities and subtraction of quantities for samples withdrawn at a particular time interval.

k. All of the differences at all time intervals are assembled in time register and displayed graphically to reveal, essentially, a motion picture of cellular changes with respect to time.

2. In more detail, this method features:

a. Two cell types to be compared that are, identical and are grown in culture. At a certain moment in time, one culture of cells is treated by addition of a quantity of drug or other substance that alters the biological activity of that cell type. The other culture of cells is left untreated, or may be treated with a placebo substance. The two cell cultures are here designated as treated (T) and untreated (U).

b. Ten or more time periods at which samples of T and U are withdrawn from culture for determination of the identities of all proteins present and their corresponding quantities. Thus, T and U at these times will be designated as T₁₋₁₀ and U₁₋₁₀.

c. A device or pair of devices for gentle and rapid disruption (“lysis”) of samples of T and U, such as a French Pressure Cell™ made by Thermo Spectronic. At each time point, the two cellular lysates of T and U may then be transferred to fractionation devices described below.

d. Approximately five kinds of fractions created from cell samples T₁₋₁₀ and U₁₋₁₀. These fractions are designated as T₁₋₁₀F₁₋₅, and U₁₋₁₀F₁₋₅.

e. Approximately five kinds of subfractions created from each of the fractions of T₁₋₁₀F₁₋₅, and U₁₋₁₀F₁₋₅. These subfractions are designated as T₁₋₁₀F₁₋₅S₁₋₅, and U₁₋₁₀F₁₋₅S₁₋₅.

f. A total of 500 sample preparation vessels such as microplates bearing labels that may be scanned or read by eye. The labeling scheme is T₁₋₁₀F₁₋₅S₁₋₅, and U₁₋₁₀F₁₋₅S₁₋₅.

g. A total of 500 additional sample preparation vessels such as microplates in which to perform preparatory steps upon each protein subfractions prior to MS analysis of constituent peptides. In these plates such steps as reduction, carboxymethylation, and trypsinization, and separation of the proteins take place. These plates bear labels according to a scheme such as Prepared (T₁₋₁₀F₁₋₅S₁₋₅), and Prepared (U₁₋₁₀F₁₋₅S₁₋₅)

h. An unknown number of proteins are in each subfraction. As the proteins are identified upon mass spectrometric identification of their constituent peptides, the proteins are designated as T₁₋₁₀F₁₋₅S₁₋₅, P₁₋ₙ, and U₁₋₁₀F₁₋₅S₁₋₅, P₁₋ₙ, where n is the number of proteins that are found in each subfraction. Let us assume that the total number of proteins in all subfractions of T and U is about 5000. Thus n is likely to reach a number of about 200 for each subfraction of T and U.

i. An unknown number of structural states for each protein (e.g. phosphorylated and nonphosphorylated). The variation in structure for each protein may then be designated as T₁₋₁₀F₁₋₅S₁₋₅, P₁₋ₙ, D₁₋ₘ, and U₁₋₁₀F₁₋₅S₁₋₅, P₁₋ₙ, D₁₋ₘ.

j. An unknown number of peptides rendered from all of the proteins present in each subfraction. These peptides are mass analyzed as the basis for determining the identity and relative quantity of each of the proteins in T₁₋₁₀F₁₋₅S₁₋₅D₁₋ₘP₁₋ₙ, and U₁₋₁₀F₁₋₅S₁₋₅D₁₋ₘP₁₋ₙ.

k. Twenty-five mass spectrometry systems capable of tandem (or higher order) mass spectrometry of peptides. Each mass spectrometer is configured to as to:

i. Access its fractional share of the subsamples described; either directly upon an autosampling stage, or indirectly by manual or automated re-supply of an unautomated stage.

ii. Perform one or two dimensional microcapillary HPLC to separate the constituent peptide mixture prior to MS analysis. (Yates)

iii. Perform at least tandem mass spectrometry on the peptide fragments so as to enable the positive identification of the protein from which each peptide derives (Yates)

iv. Execute peptide mass-mapping identification of proteins according to the Yates method, using the SEQUEST or Turbo-SEQUEST™ software configured on a computer.

A preferred system includes:

3. One sample-preparation device configured to achieve automated fractionation of samples T₁₋₁₀ and U₁₋₁₀. Each fractionation generates approximately five fractions. For example a microplate magnetic particle processor (MMPP) such as the Thermo Labsystems Kingfisher may be used in conjunction with appropriately derivatized magnetic particles to achieve magnetic fractionation of T₁₋₁₀ and U₁₋₁₀ One MMPP device is sufficient for this function because the MMPP can process two plates at once. Moreover, the Kingfisher ML™ is a system adapted to fractionating milliliters of extract at a time, so that there is sufficient product from a single fractionation procedure to feed all five subfractionation procedures that follow. The five types of magnetic particles that achieve the five dimensions of initial fractionation could include, but is not limited to magnetic particles with covalently or noncovalently attached:

a. Antibodies that specifically bind to membrane embedded proteins.

b. Chromatographic moieties such as strong anions, strong cations, and hydrophobic groups.

4. Five additional MMPP devices configured to create approximately five subfractions out of each fraction. Five is the required number for this example and allows T and U samples to be processed at the same time. Again, the MMPP can process two plates at once. The five types of magnetic particles that achieve the five dimensions of initial fractionation could include, but is not limited to magnetic particles with covalently or noncovalently attached:

a. Antibodies that specifically bind to soluble or membrane embedded proteins.

b. Chromatographic moieties such as strong anions, strong cations, and hydrophobic groups.

c. Enzymatic substrates and structural analogs thereof.

5. A liquid handling device that is configured to distribute the liquid product of fractionation to the five microplates in which subfractionation will then take place.

6. A liquid handling device that is configured to fill the 500 microplates with appropriate quantities of appropriate reagents, buffers, magnetic particles and other materials needed according to the labeled identity of the microplate, and the particularities of the fractionation or subfractionation protocol to which they will be submitted.

7. A bar code scanner or other device for tracking the identity of microplates as they are transferred from instrument to instrument, and software configured to track said transfers.

8. A sample transfer system (STS) to move microplates among and between the devices and instruments described above in an automated fashion. This system may be comprised of a robotic microplate handling robot positioned or enabled to “reach” each of the instruments and devices in consideration.

9. A Central Processor (CP), which is a computer that is configured so as to:

a. Construct a virtual model of the overall experiment by assigning the meaningful identities to each of the samples (e.g. T₁₋₁₀F₁₋₅S₁₋₅, and U₁₋₁₀F₁₋₅S₁₋₅) upon user request within a graphical user interface.

b. Construct a database matrix of T₁₋₁₀F₁₋₅S₁₋₅D₁₋ₘP₁₋ₙ, and U₁₋₁₀F₁₋₅S₁₋₅D₁₋ₘP₁₋ₙ, that will be populated over the course the experiment by the data describing the identity and abundance of peptide that is mass analyzed by tandem MS.

c. Generate appropriate labels for microplates and or experiment maps to guide the correct placement and order of the 500 microplates (this example) containing 10 kinds of beads, buffers with respect to the robotic plate handlers and or sample preparation devices.

d. Be connected with the STS so as to send sample transfer instructions to the robotic processors and receive information about the identity of any microplate that is being transferred.

e. Be connected with the sample preparation devices (e.g. Kingfisher™ instruments) so as to send processing instructions to the robotic handler and receive information about the identity of any microplate that undergoes processing on the devices.

f. Be connected with the bar code scanners so as to record the location of any microplate if it changes physical position.

g. Be connected with the 25 computers that are running the 25 mass spectrometry systems so as to draw individual peptide identity/abundance measurements from each mass spectrometry system into the appropriate data-cell of the matrix described in part 16b.

h. Perform any necessary alignment of protein identities in the D₁₋ₘP₁₋ₙ matrix for both T and U in the event that the same proteins are present in the T and U portions of the matrix, but they are not in the same discovered order. Such alignment of identities in the matrix are necessary in order to subtract the elements of the T₁₋₁₀F₁₋₅S₁₋₅ D₁₋ₘP₁₋ₙ matrix, from the corresponding elements of U₁₋₁₀F₁₋₅S₁₋₅D₁₋ₘP₁₋ₙ matrix. This subtraction is the goal of the whole experiment because it reveals changes in protein composition that result specifically from the treatment applied (i.e. “T”).

i. Perform the subtraction of any and all quantities of all entities of the T₁₋₁₀F₁₋₅S₁₋₅D₁₋ₘP₁₋ₙ matrix from the corresponding entities in the U₁₋₁₀F₁₋₅S₁₋₅D₁₋ₘP₁₋ₙ matrix, or the other way around (i.e. T-U or U-T). This will reveal differences in any level of the matrix for the purpose of recognizing cellular changes specifically related to the treatment (i.e. “T”).

j. Display any dimension of difference between U and T matrices in a graphical user interface that may be searched, queried, or filtered so as to suppress comparative graphical features that are uninteresting, and to focus on comparative graphical features of particular interest.

Still further embodiments are in the following claims.

Appendix

Aebersold, R., B. Rist, et al. (2000). “Quantitative proteome analysis: methods and applications [In Process Citation].” Ann N Y Acad Sci 919: 33-47.

Bennick, A. and F. Brosstad (1993). “A rapid method for selecting specific hybridoma clones using paramagnetic Dynabeads.” Scand J Immunol 38(3): 212-4.

Charlwood, J., J. M. Skehel, et al. (2000). “Analysis of N-linked oligosaccharides released from glycoproteins separated by two-dimensional gel electrophoresis.” Anal Biochem 284(1): 49-59.

Cordwell, S. J., A. S. Nouwens, et al. (2000). “Subproteomics based upon protein cellular location and relative solubilities in conjunction with composite two-dimensional electrophoresis gels.” Electrophoresis 21(6): 1094-103.

Corthals, G. L., V. C. Wasinger, et al. (2000). “The dynamic range of protein expression: a challenge for proteomic research.” Electrophoresis 21(6): 1104-15.

Dieden, R., R. K. Verbeeck, et al. (1999). “Isolation, identification and immunosuppressive activity of SDZ-IMM-125 metabolites from human liver microsomes.” Eur J Drug Metab Pharmacokinet 24(1): 83-90.

Gatlin, C. L., J. K. Eng, et al. (2000). “Automated identification of amino acid sequence variations in proteins by HPLC/microspray tandem mass spectrometry.” Anal Chem 72(4): 757-63.

Gebhardt, K., V. Lauvrak, et al. (1996). “Adhesive peptides selected by phage display: characterization, applications and similarities with fibrinogen.” Pept Res 9(6): 269-78.

Hayden, J. B., A. L. McCormack, et al. (1996). “Analysis of naturally processed peptides eluted from HLA DRB1*0402 and *0404.” J Neurosci Res 45(6): 795-802.

Islam, D. and A. A. Lindberg (1990). “Detection of Shigella dysenteriae type 1 and Shigella flexneri in feces by immunomagnetic isolation and polymerase chain reaction.” J Clin Microbiol 30(11): 2801-6.

Jackson, C. J., P. K. Garbett, et al. (1990). “Binding of human endothelium to Ulex europacus I-coated Dynabeads: application to the isolation of microvascular endothelium.” J Cell Sci 96(Pt 2): 257-62.

Kandzia, J., W. Scholz, et al. (1984). “Magnetic albumin/protein A immunomicrospheres. I. Preparation, antibody binding capacity and chemical stability.” J Immunol Methods 75(1): 31-41.

Kvalheim, G., O. Fodstad, et al. (1987). “Elimination of B-lymphoma cells from human bone marrow: model experiments using monodisperse magnetic particles coated with primary monoclonal antibodies.” Cancer Res 47(3): 846-51.

Link, A. J., J. Eng, et al. (1999). “Direct analysis of protein complexes using mass spectrometry.” Nat Biotechnol 17(7): 676-82.

Meeusen, S., Q. Tieu, et al. (1999). “Mgm101p is a novel component of the mitochondrial nucleoid that binds DNA and is required for the repair of oxidatively damaged mitochondrial DNA.” J Cell Biol 145(2): 291-304.

Morel, V., R. Poschet, et al. (2000). “Towards the proteome of the rhodopsin-bearing post-Golgi compartment of retinal photoreceptor cells [In Process Citation].” Electrophoresis 21(16): 3460-9.

Owen, C. S. and J. G. Lindsay (1983). “Ferritin as a label for high-gradient magnetic separation.” Biophys J 42(2): 145-50.

Ozyhar, A., M. Gries, et al. (1990). “Magnetic DNA affinity purification of ecdysteroid receptor.” J Steroid Biochem Mol Biol 43(7): 629-34.

Panigrahi, A. K., S. P. Gygi, et al. (2001). “Association of two novel proteins, TbMP52 and TbMP48, with the trypanosoma brucei RNA editing complex [In Process Citation].” Mol Cell Biol 21(2): 380-9.

Saleh, A., D. Schieltz, et al. (1998). “Tra1p is a component of the yeast Ada.Spt transcriptional regulatory complexes.” J Biol Chem 273(41): 26559-65.

Santoni, V., P. Doumas, et al. (1999). “Large scale characterization of plant plasma membrane proteins.” Biochimie 81(6): 655-61.

Santoni, V., T. Rabilloud, et al. (1999). “Towards the recovery of hydrophobic proteins on two-dimensional electrophoresis gels.” Electrophoresis 20(4-5): 705-11.

Scheffold, A., S. Miltenyi, et al. (1995). “Magnetofluorescent liposomes for increased sensitivity of immunofluorescence.” Immunotechnology 1(2): 127-37.

Simpson, R. J., L. M. Connolly, et al. (2000). “Proteomic analysis of the human colon carcinoma cell line (LIM 1215): development of a membrane protein database [In Process Citation].” Electrophoresis 21(9): 1707-32.

Soskic, V., M. Gorlach, et al. (1999). “Functional proteomics analysis of signal transduction pathways of the platelet-derived growth factor beta receptor.” Biochemistry 38(6): 1757-64.

Spahr, C. S., S. A. Susin, et al. (2000). “Simplification of complex peptide mixtures for proteomic analysis: reversible biotinylation of cysteinyl peptides [In Process Citation].” Electrophoresis 21(9): 1635-50.

Taylor, R. S., C. C. Wu, et al. (2000). “Proteomics of rat liver Golgi complex: minor proteins are identified through sequential fractionation [In Process Citation].” Electrophoresis 21(16): 3441-59.

te Heesen, S., R. Rauhut, et al. (1991). “An essential 45 kDa yeast transmembrane protein reacts with anti-nuclear pore antibodies: purification of the protein, immunolocalization and cloning of the gene.” Eur J Cell Biol 56(1): 8-18.

Ushijima, H., H. Honma, et al. (1990). “Removal of HIV antigens and HIV-infected cells in vitro using immunomagnetic beads.” J Virol Methods 29(1): 23-31.

Yanagida, M., Y. Miura, et al. (2000). “Matrix assisted laser desorption/ionization-time of flight-mass spectrometry analysis of proteins detected by anti-phosphotyrosine antibody on two-dimensional-gels of fibrolast cell lysates after tumor necrosis factor-alpha stimulation [In Process Citation].” Electrophoresis 21(9): 1890-8.

Yates, J. R., 3rd, E. Carmack, et al. (1999). “Automated protein identification using microcolumn liquid chromatography-tandem mass spectrometry.” Methods Mol Biol 112: 553-69.

Yates, J. R., 3rd, A. L. McCormack, et al. (1997). “Direct analysis of protein mixtures by tandem mass spectrometry.” J Protein Chem 16(5): 495-7.

Zhang, W., A. J. Czernik, et al. (1994). “Matrix-assisted laser desorption mass spectrometric peptide mapping of proteins separated by two-dimensional gel electrophoresis: determination of phosphorylation in synapsin I.” Protein Sci 3(4): 677-86.

What is claimed is:

1. A method for analysis of proteins in a biological system comprising:
providing a biological system;
exposing the system to a stimulus;
sampling the biological system at multiple time intervals after exposing the system to the stimulus,
treating the multiple samples by separation technique to provide multiple protein samples suitable for analysis by mass spectrometry, and
analyzing the multiple samples to determine changes in protein abundance as a function of time after exposing the biological system to stimulus, said analyzing including
providing a parallel array of mass spectrometry systems adapted for protein analysis, and
directing mass spectral data from the mass spectrometry systems in said array to a common computing device, said mass spectral data being indicative of the identity and the abundance of protein in said multiple sample, and
correlating said mass spectral data as a function of time.

2. The method of claim 1 comprising displaying said correlated data as a function of protein identity, protein abundance, and time.

3. The method of claim 1 wherein the correlated data is stored in a searchable database.

4. The method of claim 1 comprising identifying proteins based on changes in abundance as a function of time.

5. The method of claim 4 wherein said array includes at least 20 mass spectrometers.

6. The method of claim 4 comprising analyzing 500 proteins or more.

7. The method of claim 6 comprising analyzing 5000 proteins or more.

8. The method of claim 4 wherein the separation technique includes separation apparatus and said common computing device communicates with said separation apparatus.

9. The method of claim 8 wherein the separation technique includes chromatography.

10. The method of claim 8 wherein the separation technique includes use of a magnetic particle separation apparatus.

11. The method of claim 10 where the magnetic particle separation apparatus treats multiple samples in parallel.

12. The method of claim 4 wherein said mass spectral data includes peptide fragment mass spectra and an amino acid sequence derived from a data base.

13. The method of claim 12 wherein said mass spectrometer are LC-TMS mass spectrometers.

14. The method of claim 4 comprising
exposing a first component of the biological system to a stimulus and maintaining a second component of the biological system free of the stimulus,
sampling and analyzing each of the first component and the second component and comparing the identity and abundance in the first component and the second component.

15. The method of claim 14 comprising separately analyzing samples from said first component and second component.

16. The method of claim 4 wherein the stimulus is a drug.

17. The method of claim 4 wherein the time interval is about 5 to 60 seconds.

18. The method of claim 4 wherein the time interval is about one minute to one hour.

19. A system for mass spectrometric analysis comprising:
a parallel sample separation apparatus adapted to separate multiple samples in parallel for analysis by mass spectrometry, and
a parallel array of mass spectrometry systems adapted to receive the samples from the separation apparatus, and
a common computing device communicating with the parallel array of mass spectrometry systems and the parallel separation apparatus, the common computing device being adapted to analyze mass spectral data from the parallel array of mass spectrometry systems as function of sample identity.

20. The system of claim 19 where the parallel separation device is a parallel magnetic particle separation device.

21. The system of claim 19 wherein said array includes at least 2 mass spectrometers.

22. A method for analysis of proteins in a biological system comprising:
providing a biological system containing proteins;
exposing the biological system to a stimulus;
after exposing the biological system to the stimulus, sampling the biological system at multiple time intervals to obtain multiple samples;
treating the multiple samples by a separation technique to provide multiple protein samples suitable for analysis by mass spectrometry;
providing a parallel array of mass spectrometer systems capable of simultaneous analysis of as many protein samples as there are spectrometer systems in said array;
analyzing the multiple protein samples in said parallel array of mass spectrometry systems to generate mass spectral data indicative of the identity and the abundance of proteins in said multiple protein samples; and
in a common electronic computing device communicating with each of said mass spectrometry systems, correlating said mass spectral data as a function of time.

23. The system of claim 22 where the parallel separation device is a parallel magnetic particle separation device.

24. The system of claim 23 wherein the parallel array includes an array of LC-MS spectrometer system.

25. The system of claim 24 wherein the array includes 6-20 mass spectrometers.

26. The system of claim 25 wherein the time intervals are in the range of 5 seconds to 10 minutes.

27. The system of claim 26 wherein the analysis includes analysis of about 500 proteins or more.

28. The method of claim 27 wherein the central computer communicates with the separation.
